(19) United States
(12) Patent Application Publication
(10) Pub. No.: US 2003/0078545 A1
(43) Pub. Date: Apr. 24, 2003

(54) System for administering a drug delivery device

(76) Inventors: Janet Howie, Keller, TX (US); Kerry Markwardt, Mansfield, TX (US); Theron R. Rodstrom, Cranfills Gap, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/037,345

(22) Filed: Oct. 23, 2001

(51) Int. Cl. A61M 5/00

(52) U.S. Cl. 604/232

(57) ABSTRACT

A system having an injection cartridge and plunger tip which are adapted to be used on commercially available intraocular lens injection systems. The injection cartridge is shorter and smaller in diameter than typical intraocular lens injection cartridge. The plunger tip has an extended, small diameter section sized to slide easily within a round bore in the cartridge. A stop prevents the plunger tip from being expelled from the cartridge. The entire system, including drug delivery device, may be packaged as a single, terminally sterilized unit, does not require manipulation of the drug delivery device and may be used with a wide variety of drug delivery formulations.

This invention relates to intraocular drugs and more particularly to devices used to administering drug delivery devices into an eye.

### BACKGROUND OF THE INVENTION

Various solid and semi-solid drug delivery implants are known, including both non-erodible, non-degradable implants, such as those made using ethylene vinyl acetate, and erodible or biodegradable implants, such as those made using polyanhydrides or polylactides. Drug delivery implants, particularly ophthalmic drug delivery implants are generally characterized by at least one polymeric ingredient. In many instances, drug delivery implants contain more than one polymeric ingredient.

For example, U.S. Pat. No. 5,773,019 discloses implantable controlled release devices for delivering drugs to the eye wherein the implantable device has an inner core containing an effective amount of a low solubility drug covered by a non-bioerodible polymer coating layer that is permeable to the low solubility drug.

U.S. Pat. No. 5,378,475 discloses sustained release drug delivery devices that have an inner core or reservoir comprising a drug, a first coating layer which is essentially impermeable to the passage of the drug, and a second coating layer which is permeable to the drug. The first coating layer covers at least a portion of the inner core but at least a small portion of the inner core is not coated with the first coating layer. The second coating layer essentially completely covers the first coating layer and the uncoated portion of the inner core.

U.S. Pat. No. 4,853,224 discloses biodegradable ocular implants comprising microencapsulated drugs for implantation into the anterior and/or posterior chambers of the eye. The polymeric encapsulating agent or lipid encapsulating agent is the primary element of the capsule.

U.S. Pat. No. 5,164,188 discloses the use of biodegradable implants in the suprachoroid of an eye. The implants are generally encapsulated. The capsule, for the most part, is a polymeric encapsulating agent. Material capable of being placed in a given area of the suprachoroid without migration, “such as oxycel, gelatin, silicone, etc.” can also be used.

These devices must all be placed in the eye in a proper and stable location. Prior to the present invention, these devices were implanted into the eye using a pair of forceps and manipulated to the correct location using a surgical hook. Such a technique increases the chances for tissue damage and may compromise sterility of the implant.

Accordingly, a need continues to exist for an accurate, safe and convenient device for positioning intraocular drug delivery implants

### BRIEF SUMMARY OF THE INVENTION

The present invention improves upon the prior art by providing a system having an injection cartridge and plunger tip which are adapted to be used on commercially available intraocular lens injection systems. The injection cartridge is shorter and smaller in diameter than typical intraocular lens injection cartridge. The plunger tip has an extended, small diameter section sized to slide easily within a round bore in the cartridge. A stop prevents the plunger tip from being expelled from the cartridge. The entire system, including drug delivery device, may be packaged as a single, terminally sterilized unit, does not require manipulation of the drug delivery device and may be used with a wide variety of drug delivery formulations.

Accordingly, an objective of the present invention to provide a system for locating a drug delivery device into the eye.

It is a further objective of the present invention to provide a system for locating a drug delivery device into the eye that does not require direct manipulation of the device.

It is a further objective of the present invention to provide a system for locating a drug delivery device into the eye that may be packaged as a single, terminally sterilized unit.

Other objects, features and advantages of the present invention will become apparent with reference to the drawings, and the following description of the drawings and claims.

### BRIEF DESCRIPTION OF THE DRAWINGS

FIG. 1 is a perspective view of the cartridge used with the system of the present invention.

FIG. 2 is a longitudinal cross-sectional view of the cartridge shown in FIG. 1.

FIG. 3 is a perspective view of the plunger tip used with the system of the present invention.

FIG. 4 is an exploded perspective view of the system of the present invention.

FIG. 5 is a perspective view of the system of the present invention loaded with a drug delivery device and with the cartridge shown in phantom.

FIG. 6 is a perspective view similar to FIG. 5 except showing the drug delivery device being expelled from the cartridge.

### DETAILED DESCRIPTION OF THE INVENTION

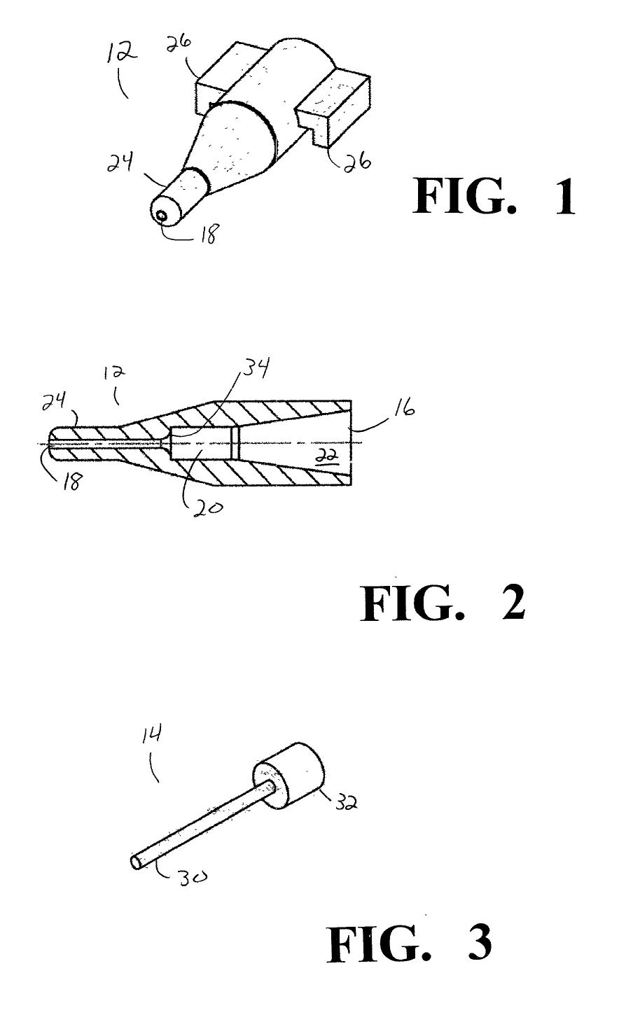

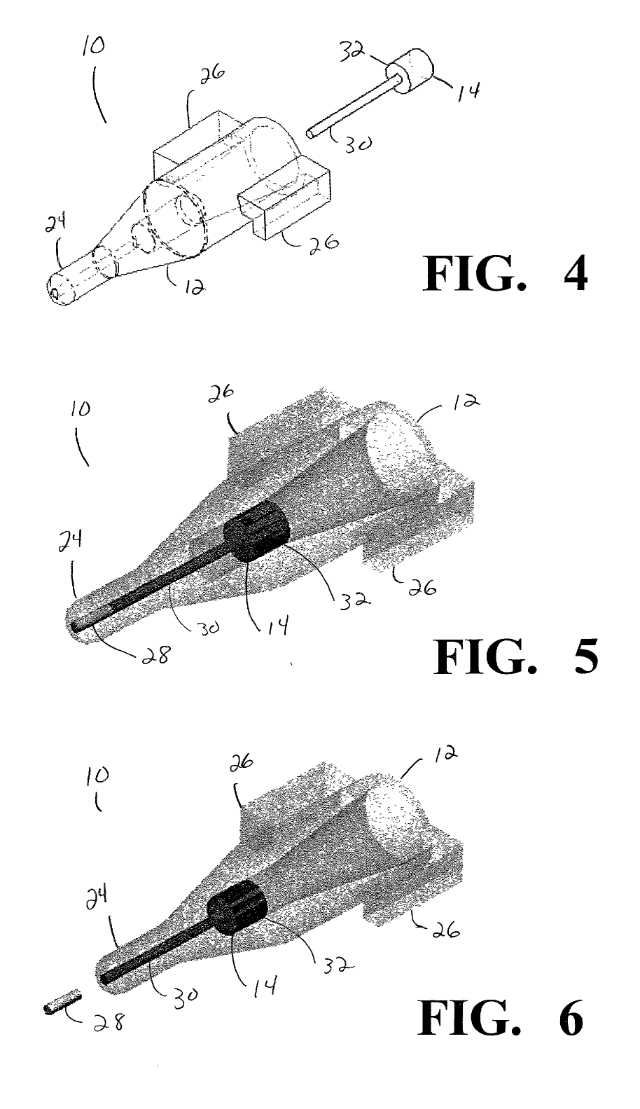

As best seen in FIGS. 1-4, system 10 of the present invention generally includes cartridge 12 and plunger tip 14. Cartridge 12 may be may of any suitable material, such as a thermoplastic. The exterior of cartridge 12 may be of any suitable size and shape suitable for use in the desired intraocular lens injection system handpiece (not shown). Suitable cartridge exterior shapes and handpieces are generally disclosed in U.S. Pat. Nos. 5,947,976, 6,010,510 and 6,083,231, the entire contents of which being incorporated herein by reference. Cartridge 12 contains a bore 16 having a narrow cylindrical portion 18, a larger cylindrical portion 20, and a flared portion 22. Narrow portion 18 is contained within injection nozzle 24, which comprises the distal end of cartridge 12. Nozzle 24 is sized and shaped so as to be able to deposit drug delivery device 28 near delicate ophthalmic tissue, for example, the anterior chamber angle. Such size and shape is generally shorter and smaller in diameter than currently available intraocular lens injection cartridges. Although nozzle 24 is illustrated as being straight, nozzle 24 may also be bent at any desired angle in order to facilitate the delivery of drug delivery device 28 to the desired region of the eye. Cartridge 12 may also contain grips 26 that allow for hand manipulation of cartridge 12 and provide a way for holding cartridge 12 within the handpiece. Plunger tip 14 generally contains injecting rod 30 and stop 32. As best seen in FIGS. 2, 4 and 6, rod 30 is sized and shaped to slide easily within narrow portion 18 of nozzle 24, but is prevented from extending beyond nozzle 24 by stop 32 contacting shoulder 34 at the intersection of narrow portion 18 and larger portion 20. Plunger tip 14 may be made from any suitable material, such as silicone rubber or other elastomer, and stop 32 may be any shape suitable for use with the selected handpiece and plunger rod.

As best seen in FIGS. 5-6, in use, system 10 of the present invention is shipped with plunger tip 14 and drug delivery device 28 installed within bore 16, for example, by forming device 28 outside of bore 16 and insert device 28 into bore 16 or by forming device 28 in bore 16 by injecting device 28 into bore 16 in a semi-solid state and allowing device 28 to solidify within bore 16. Prior to shipment, system 10 may be aseptically packaged and terminally sterilized. To expel drug delivery device 28, system 10 is installed in a suitable intraocular lens injection system handpiece (not shown but well-known in the art) and the handpiece is operated in a manner well-known in the art so that the handpiece plunger rod contacts stop 32 and pushes rod 30 and drug delivery device 28 forward, eventually expelling drug delivery device 28 out of nozzle 24.

While certain embodiments of the present invention have been described above, these descriptions are given for purposes of illustration and explanation. Variations, changes, modifications and departures from the systems and methods disclosed above may be adopted without departure from the scope or spirit of the present invention.

We claim:

1. A system for administering a drug delivery device, comprising:
a) a cartridge having a nozzle and a bore;
b) a plunger tip sized and shaped to fit within the bore of the cartridge; and
c) a drug delivery device sized and shaped to fit within the bore of the cartridge.

2. The system of claim 1 wherein the drug delivery device is formed within the bore.

3. A system for administering a drug delivery device, comprising:
a) a cartridge having a nozzle and a bore;
b) an elastomeric plunger tip sized and shaped to fit within the bore of the cartridge; and
c) a drug delivery device formed within the bore of the cartridge.
